(19) United States
(12) Patent Application Publication
(10) Pub. No.: US 2005/0214809 A1
(43) Pub. Date: Sep. 29, 2005

(54) Real-time detection of nucleic acids and proteins

(76) Inventor: Myun Ki Han, Clarksville, MD (US)

Correspondence Address: WHITEFORD, TAYLOR & PRESTON, LLP;ATTN: GREGORY M STONE, SEVEN SAINT PAUL STREET, BALTIMORE, MD, 21202-1626, US

(21) Appl. No.: 10/997,674

(22) Filed: Nov. 24, 2004

(30) Foreign Application Priority Data
Nov. 25, 2003 (KR) 10-2003-0084116

(51) Int. Cl. C12Q 1/68

(52) U.S. Cl. 435/6

(57) ABSTRACT

The present invention provides a method for real-time detection of an independent target nucleic acid or target nucleic acid linked to a secondary structure through signal amplification (direct detection) or through detection of the target nucleic acid sequence which has been the subject of an amplification process. A probe including a detectable marker is hybridized to either an independent target nucleic acid or a linked target nucleic acid to provide verification of the presence of the target nucleic acid and/or secondary structure to which the target nucleic acid is linked within either isothermal or non-isothermal environments of homogeneous or heterogeneous systems.

### CROSS REFERENCE TO RELATED APPLICATION

The present application claims priority under 35 U.S.C. 119(a) to the Korean Patent Application Number 10-2003-0084116, filed with the Korean Patent Office, filed on Nov. 25, 2003, which is herein incorporated by reference in its entirety.

### FIELD OF THE INVENTION

The present invention generally relates to the field of biochemistry and molecular biology, and particularly to the real-time detection of nucleic acid reactions. More particularly, the invention relates to nucleic acid probes and their methods of use in nucleic acid reactions for the detection of specific nucleic acid sequences, nucleic acid sequences attached to secondary molecules, and/or nucleic acid sequences containing single nucleotide polymorphisms.

### BACKGROUND OF THE INVENTION

Methods to specifically detect nucleic acids and proteins have become a fundamental aspect of scientific research. The ability to detect and identify certain nucleic acid regions and proteins has allowed researchers to determine what genetic and biological markers are indicative of human medical conditions. This ability has led to the development of in vitro diagnostic kits and kits to detect and identify pathogens and bio-warfare agents from environmental samples. Products in the in vitro diagnostics industry generally gall into the following methodological categories: clinical chemistry, microbiology, nucleic acid testing, cellular analysis, hematology, blood banking, hemostasis, and immunohistochemistry. These products have had wide range of application that include infectious disease, diabetes, cancer, drug testing, heart disease, and environmental testing of pathogens.

The diagnostics industry has been dominated by traditional immunochemistry test methods and targets in microbiology. However, these tests are gradually being displaced by faster and more effective molecular diagnostic tests. With the enormous amount of research focused on understanding the human genome, new targets for molecular testing are being discovered. As the abundance of information derived from the human genome begins to yield commercial diagnostic protocols, it is expected that the strongest growth may be seen in the nucleic acid testing market. Examples such as pharmacogenomic profiling and the assessment of which therapeutic drugs are best suited for patients based on their genetic makeup may become available, as millions of single-nucleotide polymorphisms (SNP's) have been identified.

Nucleic acid testing has been revolutionized by nucleic acid amplification methods. Examples of such methods are the polymerase chain reaction (PCR) (Mullis, Cold Spring Harbor Symp. Quant. Biol. 51:263-273 (1986)), strand displacement amplification (SDA) (Walker, Little, Nadeau, and Shank, Proc. Natl. Acad. Sci. USA 89:392-396 (1992), Walker, Fraiser, Schram, Little, Nadeau, and Malinowski, Nucl. Acids Res. 20:1691-1696 (1992)), ligase chain reaction (LCR) (Wu and Wallace, Genomics 4:560-569 (1989), Barany, Proc. Natl. Acad. Sci. USA 88:189-193 (1991), Barany, PCR Methods Appl. 1:5-16 (1991)) nucleic acid sequence based amplification (NASBA) (Kwoh, Davis, Whitfield, Chappelle, DiMichele, and Gingeras, Proc. Natl. Acad. Sci. USA 86:1173-1177 (1989), Guatelli, Whitfield, Kwoh, Barringer, Richman, and Gingeras Proc. Natl. Acad. Sci. USA 87:1874-1878 (1990), Compton, Nature 350:91-92 (1991)) and rolling circle amplification (RCA) (Fire, and Xu, Proc. Natl. Acad. Sci. USA 92:4641-4645 (1995), Liu, Daubendiek, Zillman, Ryan, and Kool, J. Am. Chem. Soc. 118:1587-1594 (1996), Lizardi, Huang, Zhu, Bray-Ward, Thomas, Ward, Nature Genet. 19:225-232 (1998), Baner, Nilsson, Mendel-Hartvig, and Landegren, Nucl. Acids Res. 26:5073-5078 (1998). Numerous clinical diagnostic tests currently in use or under development have been based on the extreme sensitivity that these amplification methods provide. These tests have been able to considerably reduce the time required for detection from days or weeks to hours, while maintaining the level of specificity required for diagnostic testing.

Conventional detection methods of nucleic acid amplification reactions are well known by those skilled in the art. These detection schemes are generally labor intensive post-amplification procedure, requiring electrophoresis or utilizing probing and/or blotting techniques. Examples of these types of methods are enzyme-linked gel assays, enzymatic bead based detection, electrochemiluminescent detection, fluorescence correlation spectroscopy, and microtiterplate sandwich hybridization assays, all of which have been extensively described in the literature. However, these methods are heterogeneous, require additional sample handling, are time-consuming, and prone to cross-contamination. The ability to detect products concurrently with target amplification in a homogenous closed tube system would conserve time, facilitate large-scale screening and automation, and may be less prone to cross-contamination, assets desirable in diagnostic detection.

In recent years a number of DNA diagnostic systems have been developed that enable detection of the amplified product in real time without opening the reaction vessel. These homogenous systems have been based on molecular energy transfer mechanisms such as Forster resonance energy transfer (FRET). These methods detect the amplification product by the use of hybridization probes. The most described real-time detection schemes for nucleic acid detection are for the detection of polymerase chain reactions (PCR). These schemes are based on a fluorescence probe that forms a secondary structure that is quenched when not hybridized to the target. Increases in fluorescence signals are a result of probe hybridization to each amplified product at a measured time point (Taqman (Holland, Abramson, Watson and Gelfand, Proc. Natl. Acad. Sci. USA 88:7276-7280 (1991), Heid, Stevens, Livak, and Williams, Genome Res. 6:986-994 (1996)), molecular beacon (Tyagi and Kramer, Nat. Biotechnol. 14:303-308 (1996)), scorpion primers (Whitcombe, Theaker, Guy, Brown, and Little, Nat. Biotechnol. 17:804-807 (1999)). The increases in fluorescence are the result of either unfolding of the probe upon hybridization or cleavage of the probe by Taq polymerase upon hybridization to amplified product. The detection of amplicons occurs in a one amplicon to one probe ratio. At any given cycle, one amplicon results in one probe (i.e. molecular beacon, Taqman probe, etc.) being detected by hybridization and/or by cleavage of the probe.

Real-time methods to detect nucleic acid sequence based amplification (NASBA) products concurrently with amplification using molecular beacons have also been described (Leone, van Schijndel, van Gemen, Kramer, and Schoen, Nucl. Acids. Res. 26:2150-2155 (1998)). The probes are based on Förster resonance energy transfer (FRET), labeled with a fluorescence donor and quencher at the 3′ and 5′ ends. When not hybridized to the target, the donor fluorescence is quenched due to the formation of a hairpin structure bringing the donor and quencher into close proximity. As amplification of the products occur, the probe hybridizes to the amplified target DNA sequence allowing separation of the donor from the quencher. This results in an observable fluorescence signal that can be detected in a closed-tube real-time format.

Simultaneous and homogenous strand displacement amplification (SDA) reaction and detection methods have been described utilizing fluorescence polarization (Spears, Linn, Woodard, and Walker, Anal. Biochem. 247:130-137 (1997)) or Förster resonance energy transfer (FRET), (Nadeau, Pitner, Linn, Schram, Dean, and Nycz, Anal. Biochem. 276:177-187 (1999)). In both instances, internal primers are fluorescently labeled and designed to bind central portions of the target strand. In the former, the probe is not used as an amplification primer because it lacks a nickable restriction site. Hybridization of this probe to the product results in an increase in the average rotational correlation time of the probe and forms the basis of detection. With the FRET assay the probe is extended and displaced by the extension of the upstream primer. The displaced probe then serves as a template for the downstream primer and a double stranded cleavable product is formed. This product is cleaved in both strands resulting in an increase in fluorescence intensity.

Amplified rolling circle amplification (RCA) products have been previously detected by incorporation of hapten-labeled or fluorescently labeled nucleotides, or by hybridization of fluor-labeled or enzymatically labeled complementary oligonucleotides. Thomas et al. (Thomas, Nardone, and Randall, Arch. Pathol. Lab Med. 123:1170-1176 (1999)) demonstrated sensitivity of 10 target molecules and 10⁷-fold amplification in 1 hour in a homogenous closed tube format using open circles probes, exponential RCA and Amplifluor detection probes. The reaction is quantitative when using real-time instrumentation and thus has great promise in research and diagnostic use.

With all of the aforementioned real-time schemes, there are several disadvantages: 1) the probe relies on the formation of a secondary structure to quench the donor fluorescence, thus, the melting temperature of the beacon has to be tightly controlled. This may be difficult in the case of the isothermal reactions such as nucleic acid sequence based amplification (NASBA) and rolling circle amplification (RCA). The beacon must be designed to unfold at the reaction temperature to bind to the target while maintaining a hairpin structure when not hybridized. This may result in increased difficulty in probe design and problems associated with signal-to-noise because the probe often emits background fluorescence due to unfolding of the beacon at the temperature of the reaction; and 2) The signal provided by the hybridization of the probe with the target is solely the result of target amplification. With this one-to-one hybridization ratio, the limiting factor of detection relies solely on the speed of amplification. Hence, the speed of detection is constrained by the detection limits of the fluorescence probes themselves (fmol level in general). Lower levels of agent require more time to generate sufficient levels of amplicon for detection.

Thus, there exists a need in the art for assays that amplify both the target nucleic acid and the detection signal to improve upon the speed and sensitivity of nucleic acid detection.

The ability to detect proteins is an essential aspect and the largest market in the diagnostics industry. Implications range from the early detection of biological warfare exposure to the pre-phenotypic diagnosis of disease and monitoring of treatment progress. Additionally, as a result of the various genome sequencing projects new open reading frames (ORF's) have been identified for which protein products have yet to be characterized. Commonly used methods such as 2-D gel electrophoresis and enzyme linked immunosorbant assay suffer from a lack of specificity or sensitivity, while mass spectrometry, though very sensitive, requires sophisticated instrumentation and is not currently adapted to routine or high-throughput use. In contrast, methods developed for the detection of nucleic acid sequences offer excellent speed, sensitivity, and specificity. At the present time, monoclonal antibodies are the most widely used vehicles for protein selection because of their specificity and avidity. Recently developed aptamers, small molecules which exhibit therapeutic target validation characteristics and may provide interference with enzyme activity, protein-protein interactions, and signaling cascades, show promise in this area, but producing them is currently time consuming and inexact, in comparison to the established methods of monoclonal antibody production. With antibodies providing protein discrimination, what is needed, then, is a method to generate and amplify a secondary signal associated with antigen binding. Recently, methods have been devised which combine the specificity of antigen detection with the speed and convenience of nucleic acid amplification. These schemes currently show the greatest promise in specific, low-level, protein detection. Currently, there are five high sensitivity protein detection methods that incorporate specific binding entities with amplifiable material. These methods are Immuno-Polymerase Chain Reaction (1-PCR), Immuno Detection Amplified by T7 RNA Polymerase (IDAT), Proximity Dependent DNA Ligation (PDL), Immuno Strand Displacement Amplification (1-SDA), and Immuno-Rolling Circle Amplification (1-RCA).

Immuno-Polymerase Chain Reaction (1-PCR) has been used in the detection of mumps-IgG (McKie, Samuel, Cohen, and Saunders, J. Immunol. Methods. 270:135-141 (2002)), Botulinum toxin (Wu, Huang, Lai, Huang, and Shaio, Lett. Appl. Microbiol. 5:321-325 (2001)), tumor necrosis factor (Saito, Sasaki, Araake, Kida, Yagihashi, Yajima, Kameshima, and Watanabe, Clin. Chem. 45:665-669 (1999)), and the Hepatitus B surface antigen (Maia, Takahashi, Adler, Garlick, and Wands, J. Virol. Methods 53:273-286 (1995)). This process links double stranded DNA to a detector antibody. After binding, a polymerase chain reaction (PCR) is carried out in any user-defined way to exponentially amplify a nucleic acid target, which is then quantified. The concentration of the amplified product relates directly to the original nucleic acid concentration, and indirectly to the concentration of protein initially bound by the antibody.

Immuno Detection Amplified by T7 RNA Polymerase (IDAT) is similar to Immuno-Polymerase Chain Reaction (1-PCR) in that a double stranded oligo is bound to the secondary antibody, but this oligo contains the T7 RNA polymerase promoter. Under isothermal conditions T7 RNA polymerase binds the promoter to repeatedly synthesize Ribonucleic Acid (RNA) molecules (Zhang, Kacharmina, Miyashiro, Greene, and Eberwine, Proc. Natl. Acad. Sci. USA 98:5497-5502 (2001)). This behavior results in a linear amplification dependent on the number of original templates.

Immuno Strand Displacement Amplification (I-SDA), developed by Becton Dickinson, is an isothermal sequence-specific amplification platform, which also requires double stranded Deoxyribonucleic Acid (DNA) linked to a detector antibody. SDA relies on the activities of two enzymes, an exonuclease deficient polymerase and a restriction endonuclease. Two primers and the exo-fragment of polymerase are used to generate a restriction site in the presence of a thiolated deoxynucleotide triphospate (thio-dNTP). This results in a double stranded hemiphosphorthioate restriction site, which is nicked by the restriction enzyme without cutting the complementary thiolated strand (Walker, Frasier, Schram, Little, Nadeau, and Malinowski, Nucl. Acids Res. 20:1691-1696 (1992)). Upon dissociation of the restriction enzyme, the exo-polymerase initiates DNA synthesis at the nicked primer, allowing for exponential amplification of the target while displacing the previously synthesized strand. The nicking, strand displacement, and primer hybridization cycle are continuous and generate large quantities of the desired target sequence.

Proximity Dependent DNA Ligation (PDL) differs from other methods in that nucleic acids are used in place of antibodies as the medium for antigen detection (Fredriksson, Gullberg, Jarvius, Olsson, Pietras, Gustafsdottir, Ostman, and Landegren, Nat. Biotechnol. 5:473-477 (2002)). These nucleic acids (probes) are called aptamers, which are obtained through a process of in vitro selection for high affinity to a target molecule. Standard PDL requires two aptamers that bind to different regions of the protein of interest, and a third oligonucleotide strand that serves as a hybridization sequence. Each aptamer is composed of a binding region followed by a primer site for polymerase chain reaction (PCR) and finally a segment complementary to the hybridization sequence. Upon binding, the 3′ end of one aptamer and the 5′ end of the other are brought into juxtaposition by annealing to the hybridization strand, where the two ends are annealed. Once joined, PCR is performed using the two included primer sites.

Immuno-Rolling Circle Amplification (I-RCA) can be used to replicate a circularized oligonucleotide primer with linear kinetics under isothermal conditions (Fire and Xu, Proc. Natl. Acad. Sci. USA 92:4641-4645 (1995)), Liu, Daubendiek, Zillman, Ryan, and Kool, J. Am. Chem. Soc. 118:1587-1594 (1996)). In this process a circularized template is hybridized to a single stranded primer. Upon addition of a strand displacing DNA polymerase and deoxynucleotide triphospates (dNTP's), hundreds of tandemly linked copies of the template are generated within a few minutes (Schweitzer and Kingsmore, Curr. Opin. Biotechnol. 12:21-27 (2001), Lizardi, Huang, Zhu, Bray-Ward, Thomas, and Ward, Nat. Genet. 19:225-232 (2001)). For I-RCA the 5′ end of the primer is attached to the secondary antibody, and the final extended product is attached at the 3′ end of the primer (Schweitzer, Wiltshire, Lambert, O'Malley, Kukanskis, Zhu, Kingsmore, Lizardi, and Ward, Proc. Natl. Acad. Sci. USA 97:10113-10119 (2000)).

Real-time detection schemes for the aforementioned processes have been developed. These schemes are based on the detection of increases in fluorescence signals as a result of probe hybridization to each amplified nucleic acid product at a measured time point. Therefore, although they greatly improve the sensitivity of protein detection, they have the same aforementioned disadvantages of real-time nucleic acid detection schemes in terms of limitations in probe design, optimization of speed of the reaction, and maximizing signal amplification.

Therefore, it would be desirable to provide a real-time protein detection assay that permits accurate and sensitive detection, while improving upon speed and automation capability.

### SUMMARY OF THE INVENTION

Accordingly, the present invention overcomes the disadvantages of the prior art by providing a real-time method of detecting target DNA or RNA. In a first aspect of the present invention a method is provided including forming a reaction mixture that includes the target nucleic acid and a probe under conditions which allows the probe to hybridize to a specific sequence on the target. After the target-probe complex is formed, nicking or cleaving the probe at a specific site such that probe fragments are created, the probe fragments dissociate from the target nucleic acid, and another probe is allowed to hybridize to the target. The dissociation of the probe fragments allow for their detection which allows for the detection of the target nucleic acid molecule.

It is an object of the present invention to allow for the detection of target DNA or RNA in a real-time, homogenous format wherein a reaction mixture includes a target nucleic acid and a probe under conditions wherein the target nucleic acid is amplified and said probe hybridizes to a specific sequence on the amplified product. Nicking or cleaving the probe occurs at a specific site such that probe fragments are created, the probe fragments dissociate from the target nucleic acid, and another probe is allowed to hybridize to said sequence. The dissociation of the probe fragments allow for their detection which allows for the detection of the target nucleic acid molecule.

In a second aspect of the present invention, a method for detecting a target epitope, molecular regions on the surface of antigens, such as a proteins and/or carbohydrates, is provided. The method includes forming a reaction mixture that contains an aptamer that has a high affinity and specificity for the target epitope. It is to be understood that the reaction mixture may contain at least two aptamers for binding with the epitope. The aptamer is further attached with a target nucleic acid sequence which is complementary to a probe within the reaction mixture. The probe hybridizes to the target after the binding of the aptamer with the target epitope. The probe is then cleaved resulting in the formation of probe fragments which due to their structure dissociate from the target nucleic acid allowing for their detection. The detection of the probe fragments provides the indication/detection of the presence of the target epitope.

It is an object of the present invention to link the aforementioned target nucleic acid sequence to a nucleic acid amplification method to permit detection of the eptiope. The probe hybridizes to the amplified nucleic acid product, and after being nicked or cleaved by the cleaving agent the probe forms probe fragments which dissociate from the amplified target nucleic acid sequence and allow for another probe to hybridize to said sequence. From the dissociated probe fragments the target epitope may be detected.

It is a further object of the present invention to detect the presence of target proteins and/or antigens. Utilizing a target nucleic acid sequence which may be attached with an antibody with specificity for a target protein and/or antigen, a probe is hybridized to the target nucleic acid sequence. The hybridized target-probe complex may then be contacted by a cleaving agent which cleaves the probe, the cleavage creating at least two probe fragments. The probe fragments dissociate from the target, and by implication the protein and/or antigen. It is further understood that the detection of the probe fragments provides detection of the antibody to which the target nucleic acid is attached and the probe hybridized.

In a third aspect of the present invention, a method for detecting the presence of single nucleotide polymorphisms is provided. A target nucleic acid sequence including a single nucleotide polymorphism and a probe, complementary to the target nucleic acid sequence including the single nucleotide polymorphism, are contained within a reaction mixture further including a cleaving agent and any necessary buffers. The hybridization of the probe to the target nucleic acid provides a target-probe complex which is cleaved when contacted by the cleaving agent. Probe fragments are created and the probe fragments dissociate from the target. Thus, detection of the probe fragments occurs and the existence of a single nucleotide polymorphism within the target nucleic acid sequence is verified. It is an object of the present invention to provide for the detection of single nucleotide polymorphisms by detecting the absence of probe fragments created through one of the methods of the present invention.

Still further it is an object of the present invention to provide for the detection of target nucleic acid sequences, proteins, antibodies and/or antigens, and single nucleotide polymorphisms via a fluorescence emission detection method.

Another object of the present invention is to provide for the detection of target nucleic acid sequences subjected to an amplification process. It is to be understood, that the target nucleic acid sequence, when utilized within the method of the present invention may allow for the detection of proteins, antibodies and/or antigens, and single nucleotide polymorphisms, as previously described. In this manner, there is concurrent amplification of the original target nucleic acid sequences as well as amplification of the detection signal from the probe thereby providing optimum levels of both speed and sensitivity.

It is a further object of the present invention to provide a method for decreasing the occurrence of cleavage of the probe at unwanted locations on the probe.

It is to be understood that both the foregoing general description and the following detailed description are exemplary and explanatory only and are not restrictive of the invention as claimed. The accompanying drawings, which are incorporated in and constitute a part of the specification, illustrate an embodiment of the invention and together with the general description, serve to explain the principles of the invention.

### BRIEF DESCRIPTION OF THE DRAWINGS

The numerous advantages of the present invention may be better understood by those skilled in the art by reference to the accompanying figures in which:

FIG. 1 is an illustration depicting the use of a fluorescently labeled nucleic acid probe in a method for the real-time detection of a target nucleic acid sequence in accordance with an exemplary embodiment of the present invention. The probe has been internally labeled adjacent to the cleavage site (in this case an RNase H cleavage site) with a FRET pair (a fluorescent donor and acceptor). An excess of this probe is incubated at constant temperature with RNase H. The nucleic acid probe is complementary to a specific sequence within the target DNA. Upon hybridization, double stranded complexes are formed and as result cleavage sites for RNase H are formed. RNase H cleaves the formed cleavage sites resulting in two probe fragments. Upon cleavage, the two probe fragments will dissociate from the target DNA because the fragments are not stably bound at the reaction temperature. As a result of cleavage, another fluorescently labeled nucleic acid probe can then hybridize to the target and the cleavage cycle of the reaction repeated. The dissociation of the probe fragments results in an increase in fluorescence intensity that is monitored by a fluorometer or a fluorescent plate reader;

FIG. 2 is a block diagram illustrating a method of providing detection of a target nucleic acid sequence utilizing the signal amplification method of the present invention;

FIG. 3 is a block diagram illustrating a method of providing detection of a target protein utilizing the signal amplification method of the present invention;

FIG. 4 is a block diagram illustrating a method of providing detection of a single nucleotide polymorphism within a target nucleic acid sequence utilizing the signal amplification method of the present invention;

FIG. 5 is an illustration depicting a method of detecting a target nucleic acid sequence utilizing a nucleic acid probe containing a DNA enzyme mediated cleavable sequence. The target nucleic acid sequence is subjected to an amplification process which may increase the speed and sensitivity of the detection process;

FIG. 6 is an illustration of a graph depicting the kinetics of a cleavage reaction by theromostable RNase H and fluorogenic chimeric DNA-RNA substrate in the presence of target DNA. Indicated amounts of target DNA were incubated at 50° C. in the presence of 5 units of RNase H and 10 pmol of fluorogenic probe. Reactions were monitored by fluorescence intensity using a fluorescence microplate reader;

FIG. 7 is an illustration of a graph depicting the real-time detection of PCR in the presence of a 10 pmol of fluorogenic probe and 5 units of thermostable RNase H. PCR reactions were performed in the presence of the indicated amounts of target DNA and the reactions monitored on a fluorescence microplate reader;

FIG. 8 is an illustration of a graph depicting the real-time detection of a rolling circle amplification (RCA) reaction. RCA reactions contained either undiluted (▪), 1:10 (♦), 1:10² (▴), 1:10³ (●), 1:10⁴ (□), or 1:10⁵ (⋄) dilutions of circularized RCA substrate in +29 DNA polymerase buffer, with 65 pmol primer, 500 μM dNTP's, 200 μg/ml BSA, 10 pmol probe, 2.5 units E. Coli RNaseH and 5 units φ29 DNA polymerase at 37° C. The control reaction (Δ) was performed with undiluted substrate in the absence of DNA polymerase. Reactions were monitored by fluorescence intensity on a Bio-Rad I-Cycler; and

FIG. 9 is an illustration of a graph depicting cleavage reactions to detect single base pair mismatches. 10 pmol of probe were incubated with 20 pmol of the indicated base pair mismatches in the cleavable portion of the probe. Cleavage of the probe was monitored with a fluorescence microplate reader and 5 units of thermostable RNase H at 50° C.

### DETAILED DESCRIPTION OF THE INVENTION

Reference will now be made in detail to the presently preferred embodiments of the invention, examples of which are illustrated in the accompanying drawings.

The present invention provides a method for detection of a target nucleic acid sequence, such as a target DNA or RNA. Further, the present invention provides a method for detection of various molecules, such as an epitope, protein, antigen, antibody, peptide, carbohydrate, organic or inorganic compounds, linked with a target nucleic acid. The detection method of the present invention may be accomplished through signal amplification (direct detection) or through detection of DNA which has been the subject of amplification processes. A probe including a detectable marker is hybridized to a target nucleic acid to provide verification of the presence of the target nucleic acid. The probe may further provide verification of the presence of a secondary target, such as a specific epitope, protein, antigen, antibody, carbohydrate, and the like, within either isothermal or non-isothermal environments of homogeneous or heterogeneous systems.

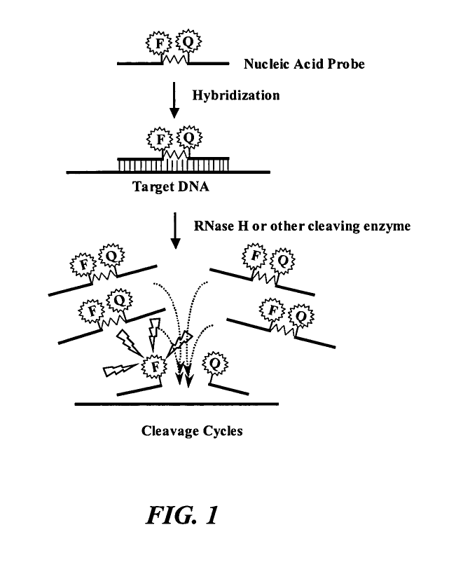

Referring generally now to FIG. 1, a method of detecting a target DNA in a real-time, homogenous format is shown. It is to be understood that the target DNA is a targeted nucleic acid sequence and may be an RNA strand without departing from the scope and spirit of the present invention. The method includes the use of a probe (nucleic acid probe) which further includes a detectable marker, for hybridization to the target DNA (target nucleic acid sequence). In the current embodiment, the detectable marker is a double label (fluorescent pair) identified as “F” (fluorescein/donor) and “Q” (acceptor/quencher). Alternatively, the detectable marker may include various identifiers and structures as will be described below. The hybridization of the nucleic acid probe with the target DNA occurs under conditions which promote a hybridization reaction or annealing of the probe with the target. The hybridization process occurs through contact by the probe with the target DNA. It is contemplated that the hybridization reaction conditions may be varied to accommodate the establishment of proper conditions for various probe and target DNA structures. The hybridization of the probe to the target DNA is followed by the cleavage of the probe, utilizing a cleaving agent (cleaving enzyme), and the dissociation of probe fragments from the target DNA. The cleaving agent contacts the probe at a cleaving site within the probe. The cleaving site may be located in various positions along the probe. For instance the cleaving site may be located proximal to the external ends of the probe, at the 5′ or 3′ end of the probe. Alternatively, the cleaving site may be located internally to the probe, more particularly within an enzyme mediated cleavable sequence of the probe which is described below. The dissociation of the probe fragments from the target DNA allows for the detection of the detectable marker. Detection occurs when the probe fragments are subjected to a detection method, such as various assay techniques, and the like, known to those of ordinary skill in the art, thereby providing indication of the presence of the target nucleic acid.

The probe may be variously constructed to accomplish its hybridization, cleavage, and dissociation functionality within the method of the present invention. In a preferred embodiment, the probe is a nucleic acid probe, formed as an oligonucleotide having a specific sequence. The specific sequence of the oligonucleotide may be predetermined or may be constructed to include a sequencing which correlates the probe with a target nucleic acid sequence. Various construction methodologies of the probe may be employed, such as those which are identified within the examples provided below, or contemplated by those of ordinary skill in the art without departing from the scope and spirit of the present invention.

The probe (nucleic acid probe), which is useful in the practice of this invention, may be constructed utilizing DNA, RNA, or a chimeric DNA/RNA nucleotide sequence. In a preferred embodiment, the probe has the structure:
R₁——X——R₂
Wherein R₁ (first probe region), R₂ (second probe region), and X (enzyme mediated cleavable sequence) are nucleic acid sequences derived from DNA, RNA, or chimeric DNA/RNA. For example, R₁ and R₂ in the nucleic acid probe may both be DNA sequences. In the alternative, R₁ and R₂ in the nucleic acid probe may both be RNA sequences. In another embodiment, the probe may include a structure in which R₁ is either RNA or DNA and R₂ is either RNA or DNA. It is to be understood that these various combinations of the R₁ and R₂ sequences may be combined with X, wherein X may be constructed of either DNA or RNA sequences. It is contemplated that R₁, R₂, and X may also be fully methylated or partially methylated to prevent non-specific cleavage.

The overall length, or number of nucleotides/base pairs, of the probe may vary to allow for the use of different target nucleic acid sequences and/or cleaving agents which are described below. It is contemplated that the length/nucleotide number of the three probe regions R₁, R₂, and X of the probe may be similarly configured, vary relative to one another, or be constructed in myriad alternative combinations with one another. For example, in one embodiment of the invention, R₁ and R₂ may be independently constructed to include one to twenty nucleotides and X may be constructed to include one to eighty nucleotides. In the alternative, R₁ may be constructed to include a sequence of one to ten nucleotides, R₂ may be constructed to include a sequence of eleven to twenty nucleotides, and X may be constructed to include a sequence of one to eighty nucleotides. In a preferred embodiment, the length of X ranges from one to ten nucleotides and more particularly from one to seven nucleotides. The length of R₁ and R₂ may be constructed ranging from one to one hundred nucleotides and more preferably from one to twenty nucleotides.

In the current embodiment, the X sequence is an enzyme mediated cleavable sequence (EMCS). Thus, the X sequence is a cleaving site of the probe allowing for the cleaving of the probe by the cleaving agent during the method of detecting the target nucleic acid of the present invention. The term “enzyme-mediated cleavage” refers to cleavage of RNA or DNA that is catalyzed by such enzymes as DNases, RNases, helicases, exonucleases, restriction endonucleases, and endonucleases. In a preferred embodiment, X is constructed of RNA and the nicking or cleaving of the hybridized probe is carried out by a ribonuclease. In still yet a further embodiment, the ribonuclease is a double-stranded ribonuclease which nicks or excises ribonucleic acids from double-stranded DNA-RNA hybridized strands. An example of a ribonuclease utilized by the present invention is RNase H. Other enzymes that may be useful are Exonuclease III and reverse transcriptase. In yet a further embodiment, the nuclease is a double stranded deoxyribonuclease that nicks or excises deoxyribonucleic acids from double stranded DNA-RNA hybridized strands. An example of a deoxyribonuclease useful in the practice of this invention is Kamchatka crab nuclease (Shagin, Rebrikov, Kozhemyako, Altshuler, Shcheglov, Zhulidov, Bogdanova, Staroverov, Rasskazov, and Lukyanov, Genome Res. 12:1935-1942 (2002)). This nuclease displays a considerable preference for DNA duplexes (double stranded DNA and DNA in DNA-RNA hybrids), compared to single stranded DNA.

In addition, due to the preferred isothermal environment within which the method of the present invention is employed, enzymes that are thermostable may increase the sensitivity, speed, and accuracy of detection. For example, the nicking or cleaving of the hybridized probe may be carried out by a thermostable RNase H. The aforementioned enzymes and others known to those of ordinary skill in the art may be employed without departing from the scope and spirit of the present invention.

The probe of the present invention may be constructed having one or more detectable markers or may link with one or more detectable markers present in a reaction mixture. It is contemplated that the detectable marker may vary, such as any molecule or reagent which is capable of being detected. For example, the detectable marker may be radioisotopes, fluorescent molecules, fluorescent antibodies, enzymes, proteins (biotin, GFP), or chemiluminescent catalysts. Fluorescent molecules and fluorescent antibodies may be termed “fluorescent label” or “fluorophore”, which herein refers to a substance or portion thereof that is capable of exhibiting fluorescence in the detectable range. Examples of fluorophores which may be employed in the present invention include fluorescein isothiocyanate, fluorescein amine, eosin, rhodamine, dansyl, JOE, umbelliferone, or Alexa fluor. Other fluorescent labels know to those skilled in the art may be used with the present invention.

The detectable marker may be a single fluorescent/fluorophore “single label” or a fluorescent pair “double label” including a donor and acceptor fluorophore, as shown in FIG. 1. The choice of single or double label may depend on the efficiency of the cleaving enzyme used and the efficiency of quenching observed. It is further contemplated that the choice of the single or double label utilized may depend on various other factors, such as the sensitivity of the detection technique (enzyme-linked gel assays, enzymatic bead based detection, electrochemiluminescent detection, fluorescence correlation spectroscopy, microtiterplate sandwich hybridization assays) being employed.

The location where the donor and acceptor fluorophores are linked with the probe may vary to accommodate the quenching capabilities of the acceptor and various other factors, such as those mentioned above. In a preferred embodiment, a double label is utilized wherein the donor and acceptor fluorophores are attached to the probe at positions which give them a relative separation of zero to twenty base pairs. More particularly the separation of the donor and acceptor is from zero to seven base pairs. This range of separation may increase the ability of the acceptor to properly quench the fluorescence of the donor until the probe is cleaved. This may further provide a reduction in the background noise experienced during the method of detection of the present invention. Thus, the signal-to-noise ratio may be maintained within optimum ranges for detection of target nucleic acid sequences.

The fluorophores may be linked with the probe at various locations and within various portions of the probe. The preferred sites of labeling are directly adjacent to X, the enzyme mediated cleavage sequence, which is preferably the cleavage site of the probe. Thus, in the current embodiment of FIG. 1, the donor is attached proximal to the 3′ end of the R₁ region of the probe also proximal to the connection of the R₁ region of the probe with the 5′ end of the X region of the probe. The acceptor is attached proximal to the 5′ end of the R₂ region of the probe which also places the acceptor in proximity to the connection of the R₂ region of the probe with the 3′ end of the X region of the probe. It is contemplated that the donor and acceptor pair, as well as any of the detectable markers which may be employed with the probe of the present invention, may be attached along the length of the R₁ and R₂ regions of the probe in relation to X. Thus, the detectable marker employed may be attached along R₁ and R₂ in positions which have varying degrees of proximity to X. Still further, the detectable markers may be externally attached at the 5′ end of the R₁ region and the 3′ end of R₂ region, respectively. Labeling of the probe with the detectable marker may also be achieved within the X region of the probe. Labeling within the X region may be preferable so long as a cleavage site is maintained in a position between probes, especially when a fluorescent pair is being employed as the detectable marker.

The detectable marker utilized and location of attachment with the probe may be dependent on the probe structure. For example, a probe constructed of a greater number of nucleotide sequences, within either the R₁, R₂, and X regions, may allow for the use of different detectable markers. Using the fluorophore pair markers as an example, a first pair of markers may include an acceptor with an increased quenching capability over an acceptor of a second pair of markers. The increased quenching capability of the first pair acceptor may allow the first pair to be separated by a larger number of nucleotides than the second pair. The greater number of base pairs between the first pair of markers may provide an advantage in the performance of the cleaving agent to cleave the probe at a cleaving site between the detectable markers. Alternatively, the ability to vary the number of base pairs between the markers may increase the performance of the hybridization of the probe with the target nucleic acid sequence.

In operation, the progression sequence shown in FIG. 1 takes place within a reaction mixture including the target nucleic acid and the probe. In forming the reaction mixture the target nucleic acid molecule and a molar excess amount of nucleic acid probe are mixed together in a reaction vessel under conditions that permit hybridization of the probe to the target nucleic acid molecule.

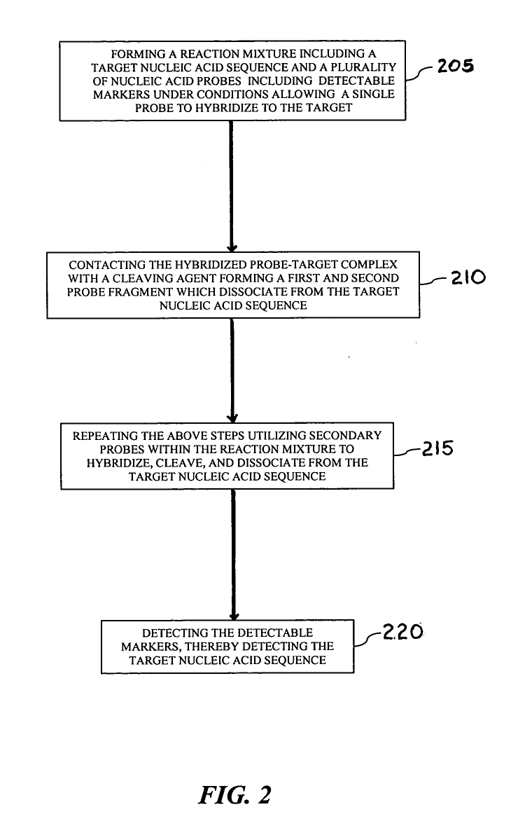

Referring now to FIG. 2, a method of detecting a target nucleic acid sequence is shown. In a first step 205 a target nucleic acid sequence is obtained. The target nucleic acid sequence may be obtained utilizing techniques and methodologies known to those of ordinary skill in the art. The target nucleic acid sequence is hybridized to a nucleic acid probe including a detectable marker forming a target-probe complex. In step 210 the target-probe complex is contacted with a cleaving agent which cleaves the probe forming probe fragments which dissociate from the target nucleic acid sequence. Steps 205 and 210 are repeated in step 215 utilizing secondary nucleic acid probes which are contained in a reaction mixture which includes the target nucleic acid sequence and a plurality of nucleic acid probes. The dissociated probe fragments allow the detectable marker to be detected which provides an indication of the presence of the target nucleic acid sequence in step 220.

In a preferred embodiment, the hybridization occurs between the probe and a specific nucleotide sequence “specific target sequence” on the target nucleic acid. This hybridization/annealing results in the formation of a double-stranded target-probe complex. The hybridized target probe complex may than be enzymatically cleaved by contacting the hybridized probe with the cleaving agent that will specifically cleave the probe at a cleaving site, which is a predetermined sequence in the hybridized probe. In a preferred embodiment, the predetermined cleavage sequence is the X region of the probe. Alternatively, the predetermined cleavage sequences may be located in various positions within the R₁ and R₂ regions of the probe.

After the enzyme-mediated nicking or cleaving of the probe at the cleaving site a first probe fragment and a second probe fragment are formed. The enzyme mediated nicking or cleaving of the probe allows the first and second probe fragments to dissociate (melt or fall off) from the target nucleic acid. The dissociation of the first and second probe fragments provide two results: (1) the detectable marker is “activated” (where a fluorescent pair is used the acceptor is displaced from the donor, freeing the donor to fluoresce) allowing for its identification through one of the various detection methods, thereby detecting the presence of the target nucleic acid sequence and (2) by dissociating from the target nucleic acid it allows another probe (secondary probe), from the molar excess of nucleic acid probes within the reaction mixture, to hybridize to the target nucleic acid at the specific target sequence. In this manner, the signal from the probe is amplified allowing for significant increases in both sensitivity and speed.

Typically, the target nucleic acid molecule and labeled probe are combined in a reaction mixture containing an appropriate buffer and cleaving agent. The reaction mixture is incubated at an optimal reaction temperature of the cleaving agent, typically in the range of 30° C. to 72° C. It is to be understood that the reaction temperature may vary based on various requirements, such as temperature requirements for various target nucleic acid molecules, temperature requirements for various nucleic acid probes, optimum performance parameters for the buffer and/or cleaving agent, and the like. The reaction mixture may be incubated from five minutes to one hundred twenty minutes to allow annealing of the probe to the target followed by subsequent cleaving of the probe. The incubation period may vary based on the various enzymes, buffers, nucleic acid sequences, and the like being utilized, which may have pre-determined optimal incubation times. As stated above, the reaction cycle involves repeating the steps of hybridization and cleavage utilizing secondary probes within the reaction mixture which react with the target nucleic acid sequence.

The cleavage or nicking of the double-stranded probe-target complex results in at least two probe fragments being formed. The fragmentation of the probe, producing reduced size probe fragments, promotes the melting or falling off of the hybridized probe fragments from the target nucleic acid under the reaction condition temperatures and permits another (secondary) probe to bind to the target. The resulting single stranded probe fragments are then identified by detection methods, thereby detecting the presence of the target nucleic acid molecule.

The identification of probe fragments may be performed using various detection methods. The method of identification and detection may depend on the type of labeling or the detectable marker incorporated into the probe or the reaction mixture. One method to detect the probe fragments is to label the probe with a Förster resonance energy transfer (FRET) pair (a fluorescence donor and acceptor). When the probe is intact, the fluorescence of the donor is quenched due to the close proximity of the acceptor. Upon physical separation of the two fluorophores, as a result of cleavage initiated by the cleaving agent, the quenched donor fluorescence is recovered as FRET is lost. Therefore, cleavage of the probe and the resulting melting away of the probe fragments results in an “activation”, increase, or recovery of donor fluorescence that may be monitored. By monitoring the increase in fluorescence, the reaction steps may be monitored in real-time thereby detecting the presence of the target nucleic acid molecule in real-time.

Modifications to the probe may also be made such that the resulting detection is only the result of specific cleavage of the X region of the probe and not due to non-specific cleavage of the R₁ and R₂ regions of the probe. For example, if the probe is a DNA-RNA-DNA chimeric probe, the DNA portion of the probe may be methylated to prevent non-specific cleavage by DNases in the reaction. Another example is if the probe is entirely constructed of RNA. The R₁ and R₂ RNA may be methylated such that only the X RNA is cleavable. Other modifications of the probe to assist in decreasing the occurrence of unwanted cleavage may be utilized as known to those of ordinary skill in the art.

The present invention also provides a method for detecting target nucleic acid sequences combined with the speed and sensitivity of nucleic acid amplification reactions. In an exemplary method a reaction mixture is formed that contains a molecule including a target nucleic acid sequence. The target nucleic acid sequence is subjected to an amplification process. A probe is included in the reaction mixture that hybridizes to the amplified target nucleic acid product. A cleaving agent nicks or cleaves the probe at a specific site such that probe fragments are formed and dissociate from the amplified target nucleic acid. The dissociation of the probe fragments allows for another (secondary) probe to hybridize to the target nucleic acid sequence. The dissociated probe fragments allow for the detection of the cleavage of the probe, thereby detecting the target nucleic acid sequence and the molecule.

In this feature of the invention, the aforementioned principles in probe design, cleavage, and detection are adapted to the detection of molecules associated with nucleic acid amplification reactions. A preferred embodiment of the invention is to use a FRET probe cleavable by RNase H along with a product molecule associated with the RCA reaction. The advantage of adapting this invention for use in conjunction with nucleic acid amplification reactions associated with various molecules is that it provides substantial improvements in the speed and sensitivity of detection.

Nucleic acid amplification reactions that are easily adaptable to this invention are well known by those skilled in the art. These reactions include but are not limited to PCR, SDA, NASBA, and RCA. In general, the target nucleic acid, probe, components of the nucleic acid amplification reaction, and a cleaving enzyme are combined in a reaction mixture that allows for the simultaneous amplification of the target nucleic acid and detection by the aforementioned cleavage of the probe. Each amplification reaction may need to be individually optimized for the respective requirements of buffer conditions, primers, reaction temperatures, and probe cleavage conditions.

The detection mechanism of the present invention may also be used for the detection of target epitopes, which may be included within various antigens, peptides, organic compounds, inorganic compounds, and the like. It is to be understood that the antigen may be various protein and/or carbohydrate substances. To accomplish the detection of a target epitope a target nucleic acid sequence that is complementary to a nucleic acid probe including a detectable marker may be attached to an aptamer that has a high affinity and specificity for the target epitope. The aptamer may be various oligonucleotides (DNA or RNA molecules) that may bind to the epitope. The aptamer may be constructed utilizing a single aptamer, a pair of aptamers, or three or more aptamers to effectively identify and bind with the target epitope. The target nucleic acid, which provides the complementary sequence, may permit the hybridization of the nucleic acid probe, forming a target-probe complex, upon the aptamer which is bound to the target epitope. The target-probe complex is subsequently cleaved and the detectable markers are detected in a manner similar to that described above, thereby detecting the presence of the target epitope.

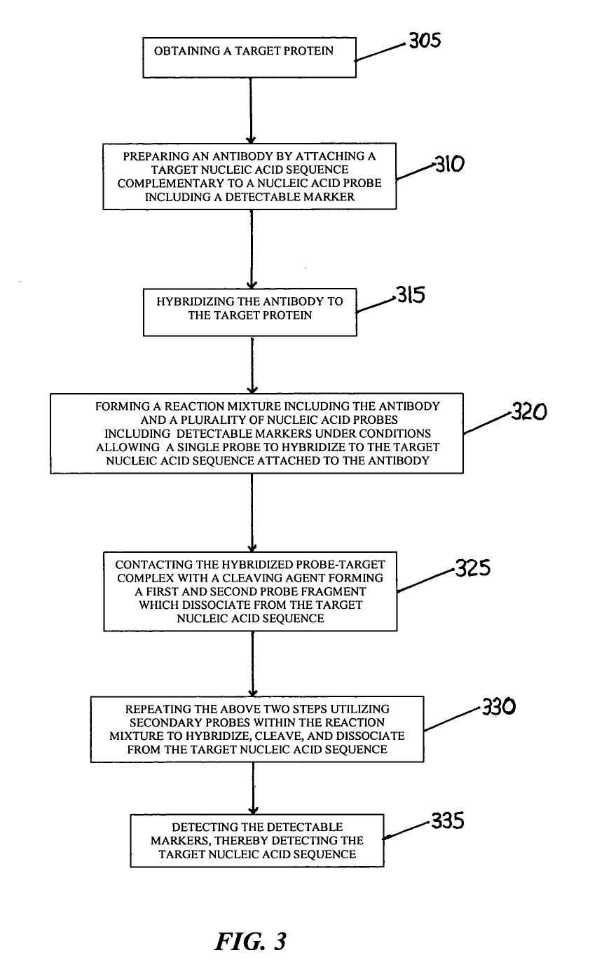

By way of example, a method of detecting a target protein is shown in FIG. 3. In a first step 305 a target protein is obtained. The target protein includes a target epitope. The obtaining of the target protein may be accomplished utilizing techniques and methodologies know to those of ordinary skill in the art. In a second step 310 an antibody which specifically targets the protein including the epitope, is prepared by attaching a target nucleic acid sequence which is complementary to a nucleic acid probe. Once the target protein is obtained and the antibody is prepared, the target protein is hybridized to the antibody in step 315 forming an antibody-target protein complex. In step 320 a reaction mixture is formed including the antibody-target protein complex and a plurality of nucleic acid probes. The plurality of nucleic acid probes each include a detectable marker and a single probe is hybridized to the target nucleic acid sequence forming a target nucleic acid-probe complex, which is attached to the antibody. A cleaving agent is provided and in step 325 the cleaving agent contacts the target nucleic acid-probe complex and cleaves the probe forming probe fragments which dissociate from the target nucleic acid. Steps 320 and 325 are repeated in step 330 utilizing secondary probes contained within the reaction mixture which hybridize, cleave, and dissociate from the target nucleic acid. In step 335 the detectable markers are detected thereby detecting the presence of the target protein. The detection of the target protein, in this manner, also provides for the detection of the antibody with which the target nucleic acid sequence was attached.

It is to be understood that the above method is exemplary and is not intended to limit the scope of the present invention. The detection of epitopes, which may be included on various structures such as antigens (proteins, carbohydrates, etc. . . . ), through the use of aptamers, antibodies, and the like may be performed utilizing a similar technique as that described above in the methods of the present invention. This detection capability may be advantageous in diagnosing the presence of various antigens possibly assisting in the providing of treatment.

The attachment of the target nucleic acid sequence to the antibody requires the design of linker nucleic acids to be attached to the 5′ end of the nucleic acids such that the hybridization sequence is not sterically hindered by the attachment to the antibody. This linker sequence is typically one to ten nucleotides, although the use of longer sequences is contemplated by the present invention. In addition, the target nucleic acid sequence may be designed to be in tandem repeats such that more than one probe can bind to each antibody, thereby amplifying the signal from each bound antibody. There are two main methods which may be used to couple the target nucleic acid sequence to the detecting antibody. In the first method 5′ thiol modified DNA is coupled to free amino groups in the antibody using either Succinimidyl-4-(N-Maleimidomethyl)Cyclohexane-1-Carboxylate (SMCC), SulfoSuccinimidyl-4-(N-Maleimidomethyl)Cyclohexane-1-Carboxylate (Sulfo-SMCC), N-Succinimidyl-3-(2-Pyridylthio)Propionate (SPDP), N-Succinimidyl-6-(3′-(2-pyridyldithio)-propionamido)hexanoate (NHS-Ic-SPDP), or SulfoSuccinimidyl-6-(3′-(2-pyridyldithio)propionaamido)hexanoate (Sulfo-NHS-Ic-SPDP). These reagents differ in the length of their spacer and degree of water solubility. If necessary, the linkage may be broken by a thiolating agent to release the DNA (target nucleic acid) for further manipulation.

In a second method, the antibody-target nucleic acid sequence bridge is supplied by the tetrameric protein strepavidin, which forms a largely irreversible bond with biotin (Niemeyer, Adler, Pignataro, Lenhert, Gao, Chi, Fuchs, and Blohm, Nucleic Acids Res. 27:4553-4561 (1999)). Free amino groups in the antibody are labeled with biotin by reaction with biotin-n-hydroxysuccinimide. Biotinylation of DNA is performed using a 5′-Biotin phosphoramidite, or by amino labeling the 5′ end, followed by reaction with biotin-n-hydroxysuccinimide. Conjugates of DNA, strepavidin, and antibody are prepared by addition of one molar equivalent of antibody to the DNA-strepavidin conjugate. After incubation for 1 hour at 4C the antibody-target nucleic acid sequence conjugate is purified on a Superdex 200 gel filtration column, where the conjugate elutes in the void volume. Samples are analyzed by non-denaturing electrophoresis on 1.5-2% agarose gels stained with Sybr-Green II.

The binding of the aptamer with the epitope or of the antibody to the target protein may occur utilizing various techniques. For example, the target protein is initially immobilized onto a solid support. Numerous methods to immobilize the target protein to the solid support are well known to those skilled in the art and may be employed without departing from the scope and spirit of the present invention. The antibody is then incubated with the immobilized target protein in a reaction mixture to allow binding of the antibody to the target protein. The bound antibody-target protein complex (including the target nucleic acid sequence attached to the antibody) is then washed several times to remove unbound antibodies. The bound antibody-target protein complex is then incubated with the aforementioned nucleic acid probe with the appropriate buffers and enzymes (cleaving agent(s)) to permit hybridization of the probe to the target nucleic acid sequence and cleavage of the probe. Detection of the cleaved probe fragments resulting from the cleaving agent contacting the probe may be accomplished through utilization of one of the aforementioned methods. The resulting dissociation of probe fragments from the target nucleic acid sequence provides the indication of the presence of the target protein.

The present invention further provides a method for detecting a target protein, antigen, epitope, and the like, that combines the speed and sensitivity of nucleic acid amplification reactions with the specificity of aptamer and/or antibody detection. In an exemplary method a reaction mixture is formed that contains a molecule such as an antibody that specifically binds to a target protein. The antibody [molecule] is attached with a target nucleic acid sequence which is linked to a nucleic acid amplification method to permit detection of antigen binding. A probe is included in the reaction mixture that hybridizes to the amplified nucleic acid product. A cleaving agent (cleaving enzyme) nicks or cleaves the probe at a specific site such that probe fragments are formed and dissociate from the amplified target nucleic acid sequence. The dissociation of the probe fragments allows for another probe to hybridize to the nucleic acid sequence. The dissociated probe fragments allow for the detection of the cleavage of the probe, thereby detecting the target protein.

In this embodiment of the invention, the aforementioned principles in probe design, cleavage, and detection are adapted to the detection of target nucleic acid sequences linked to nucleic acid amplification reactions. A preferred embodiment of the invention is to use a FRET probe cleavable by RNase H along with an antibody linked to the RCA reaction. The advantage of adapting this invention to nucleic acid amplification reactions is that it provides substantial improvements in speed and sensitivity to the specific detection of target nucleic acid sequences, which in this instance provides an advantage in detection of target epitopes, proteins, antigens, and the like.

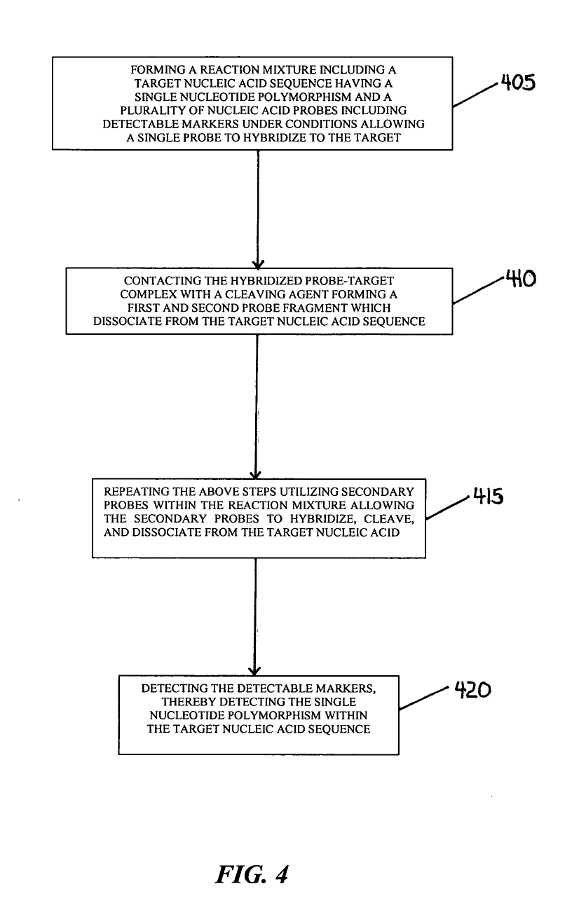

The detection of the presence of single nucleotide polymorphisms (SNP's) in target DNA may be accomplished utilizing the methods of the present invention. The labeling and detection methodology employed for detecting single nucleotide polymorphisms is similar in all respects to that employed for labeling and detecting the target nucleic acid except as described below. Referring now to FIG. 4, in a first step 405 a reaction mixture is formed containing a target nucleic acid sequence and a plurality of nucleic acid probes under conditions which allow the probe to hybridize with the target nucleic acid sequence. The target DNA includes an SNP and the probe is designed to be fully complementary with the target DNA including the complementary nucleotide matching the SNP. When contacted by a cleaving agent in step 410 the probe is cleaved into two or more probe fragments. In step 415 the steps 405 and 410 are repeated utilizing secondary probes which hybridize with the target nucleic acid sequence. The probe fragments, due to their shortened structure dissociate from the target DNA allowing a detectable marker attached with the probe to be detected in step 420. Thus, the detection of cleaved probe, in step 420, indicates the presence of the SNP within the target nucleic acid sequence.

In an alternative embodiment, an unknown SNP may be present within a target nucleic acid sequence. Thus, a probe which is complementary to the target nucleic acid sequence may present the situation where there is a single mismatch between the probe and the target nucleic acid. This mismatch, if present in the cleavable region of the probe, may not permit the probe to be cleaved by a cleaving agent. The absence of cleavage results in the absence of dissociation of probe fragments from the target nucleic acid. Thus, the target nucleic acid sequence is not ‘free’ to hybridize with secondary probes. This has the effect of limiting or canceling the production of identifiable detectable markers which are typically “activated” by their dissociation. Thus, in this embodiment it is the absence of detection of the detectable markers which indicates that there is an SNP in the target nucleic acid.

The detection of an SNP, whether by signal detection or the conspicuous absence of a signal from a detectable marker, may be performed by signal amplification, cleavage and detection of the probe itself, or in conjunction with a nucleic acid amplification reaction similar to those described previously.

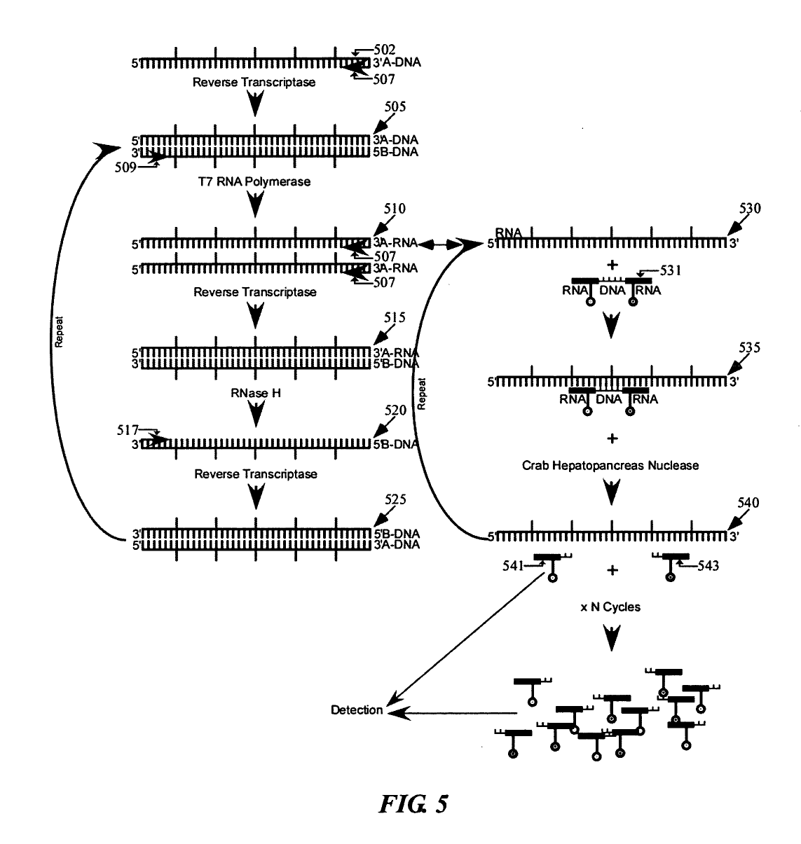

Referring now to FIG. 5, a method for detecting a target nucleic acid sequence associated with nucleic acid sequence based amplification (NASBA) is shown. In this example the probe has been internally labeled adjacent to the cleavage site (in this case an Kamchatka crab hepatopancreas duplex specific nuclease cleavage site) with a FRET pair (a fluorescent donor and acceptor) and the enzyme mediated cleavable region is composed of DNA, while the first and second probe regions are composed of RNA. In step 505 of the NASBA process a specific primer 507 is used to prime synthesis of a DNA strand complementary to the target by reverse transcriptase. The newly synthesized strand incorporates a T7 RNA polymerase promoter 509 at the 3′ end of the strand. In step 510, and in the presence of T7 RNA polymerase, the T7 promoter 509 induces production of RNA whose sequence is identical to the target, except that the product is RNA. Each T7 promoter 509 induces the production of many copies of RNA from a single template, this being the RNA amplification phase of the reaction. In step 515 copies of primer 507 bind to each RNA copy and reverse transcriptase is used to generate a double stranded RNA/DNA duplex product. In step 520 RNase H digests the RNA portion of the hybrid to generate a DNA product that is complementary to the initial target DNA. In step 525 a second primer 517 is used to prime synthesis of a DNA strand complementary to the product of step 520. This product is identical to that formed in step 505 above, thus generating more template that is further amplified during subsequent cycles of NASBA. In step 530, which begins the real-time detection phase of the reaction, a nucleic acid probe 531 complementary to the RNA products generated in step 510 hybridizes to each individual target. Upon hybridization, double stranded complexes are formed and as result cleavage sites for crab hepatopancreas nuclease are formed. In step 535 crab hepatopancreas nuclease cleaves the DNA within the formed DNA/RNA cleavage sites, resulting in a first probe fragment 541 and a second probe fragment 543. In step 540 the first probe fragment 541 and the second probe fragment 543 dissociate from the target DNA because the fragments are not stably bound at the reaction temperature, thus regenerating the initial target RNA. As a result of cleavage, another fluorescently labeled nucleic acid probe can then hybridize to the same target and the cleavage cycle of the reaction may be repeated. The advantage of adapting this invention to nucleic acid amplification reactions linked to various molecules is that it provides substantial improvements in speed and sensitivity to the specific detection of the various molecules.

Having now generally described this invention, the same will be better understood by reference to one or more specific examples. These examples are set forth to aid in the understanding and illustration of the invention, and are not intended to limit in any way the invention as set forth in the claims which follow after.

### EXAMPLE 1

Assay for Detecting Target DNA with Fluorogenic Probe and RNase H.

Preparation of Fluorescent Labeled Cleavage Probe:

A 24-mer oligonucleotide, 5′-TATGCCATTT-r(GAGA)-TTTTTGAATT-3′ (SEQ ID NO:1), was synthesized using a PerSeptive Biosystems Expedite nucleic acid synthesis system. Fluorescein and TAMRA were introduced at positions 10 and 15 by inclusion of appropriately labeled dT monomers during synthesis. Ribonucleotides, at positions 11-14, are denoted with a lowercase “r” prior to the sequence. The sialyl protecting groups on the RNA were removed by treatment overnight with tetrabutylammonium fluoride solution. An equal volume of 1M TEAA was then added to the solution followed by the addition of sterile water. The oligonucleotides were then desalted by Sephadex G-25 column. Fractions were pooled and the resulting sample was then electrophoresed on a denaturing (7M urea) 20% polyacrylamide gel to further purify the oligonucleotide and to remove any residual free dyes. The appropriate oligonucleotide band was sliced from the gel and electroeluted using the S&S ELUTRAP Electro-Separation System (Schleicher & Schuell).

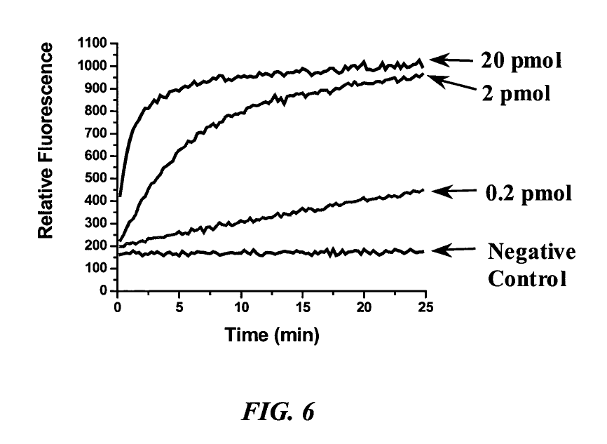

Cleavage of the probe was monitored by the increase in fluorescein emission using a fluorescence microplate reader. Different concentrations of target DNA were incubated with 10 pmol of fluorescent probe and 5 units of RNase H at 50° C. in 50 μl of 1× RNase H Buffer. The results were plotted, as shown in FIG. 6, with background subtraction of the initial relative fluorescence. A very rapid and yet distinct target dose-dependent response was observed. In as little as five minutes 0.2 pmol of target is distinguishable from the background (Negative Control). These results demonstrate that an assay from use of the method of the present invention provide extremely rapid results with statistically significant differences observed almost immediately (less than 5 minutes) for all samples. From this example it may be seen that the present invention may provide an increase in the sensitivity and speed of detection of target nucleic acids to which the probe is hybridized.

### EXAMPLE 2

Real-Time Assay for Detecting PCR Reactions with RNase H.

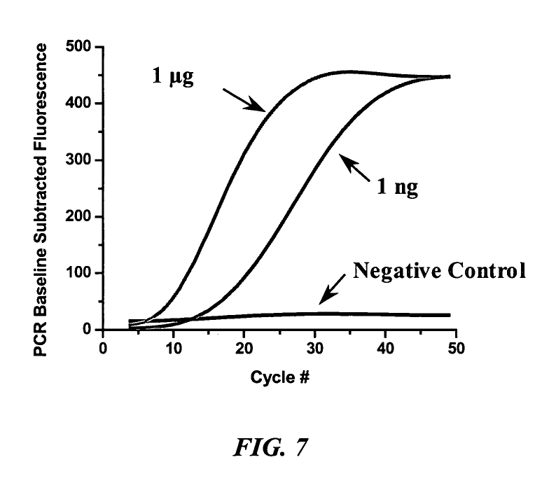

Cleavage of the probe was monitored by the increase in fluorescein emission using a fluorescence microplate reader. PCR reactions were performed with 1 μg and 1 ng of target DNA in the presence of 10 pmol of fluorescent probe and 5 units of thermostable RNase H. PCR reactions also contained 10 pmol of forward and reverse primer, 0.2 mM dNTP, and 2.5 units of Taq polymerase in 50 μl of Taq polymerase Buffer. The results, shown in FIG. 7, demonstrate that the method of the present invention may detect PCR reactions in real-time. The traces of both reactions are indicative of typical real-time PCR reactions and show similar dose dependent properties. Hence, the use of RNase H and the fluorogenic probe may provide an alternative method to real-time PCR.

### EXAMPLE 3

Simultaneous Cleavage Probe/Rolling Circle Amplification Assay to Detect DNA

Preparation of unlabeled oligonucleotides: A 60-mer oligonucleotide template, 5′-ATCTGACTATGCTTGTACCTGGTTATTTAGCACTCGTTTTTAATCAGCTCACTA GCACCT-3′ (SEQ ID NO:2), 80-mer circularizable oligonucleotide, 5′-CTAAATAACCAGGTACAATATGCCATTTGAGATTTTTGAATTGGTCTTAGAAC GCCATTTTGGCTGATTAAAAACGAGTG-3′ (SEQ ID NO:3), and 15-mer oligonucleotide primer, 5′-TGGCGTTCTAAGACC-3′ (SEQ ID NO:4), were synthesized using a PerSeptive Biosystems Expedite nucleic acid synthesis system. The oligonucleotides were purified on C18 columns.

Preparation of the rolling circle amplification substrate: An 800 uM solution of circularizable oligonucleotide was kinased in 1× T4 DNA ligase buffer containing 10 U of T4 polynucleotide kinase for 60 minutes at 37° C., followed by inactivation of the kinase for 20 minutes at 65° C. A solution containing 400 nM of this material was annealed and ligated to 200 nM template oligonucleotide in 1× T4 DNA ligase buffer containing 2000 U of T4 DNA ligase for 16 hours at 16° C.

Cleavage of the probe was monitored by the increase in fluorescein emission using a Bio-Rad I-Cycler. Fluorescein emission was base-line subtracted and well factors were collected using the experimental plate method. Intensity data were collected at one-minute intervals for the time specified. All fluorescence measurements were performed in φ29 DNA polymerase buffer (50 mM Tris-HCl, pH 7.5, 10 mM MgCl₂, 10 mM (NH₄)₂SO₄, 4 mM DTT, and contained varying concentrations of circularized RCA substrate, 65 pmol of primer, 500 μM deoxynucleoside triphosphates, 200 μg/ml BSA, 10 pmol of probe, 2.5 units of E. Coli RNaseH, and 5 units of φ29 DNA polymerase in a volume of 20 μl for 120 minutes at 37° C.

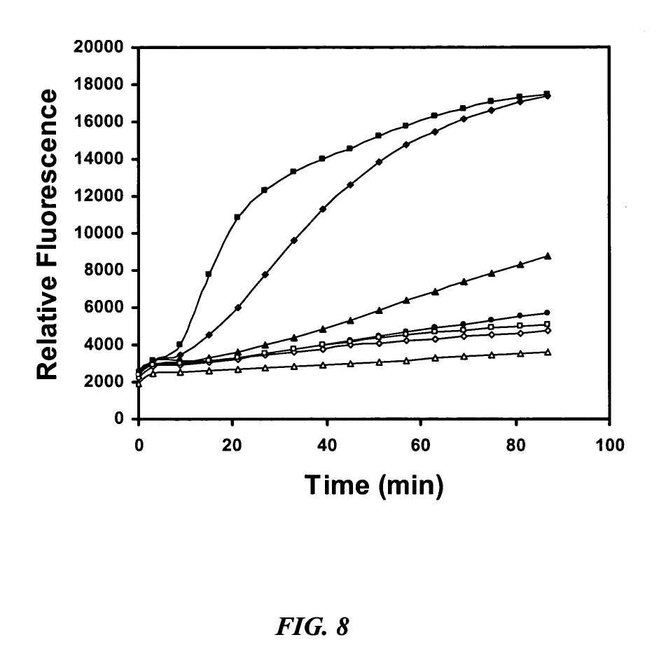

Rolling circle amplification is an isothermal technique for the rapid generation of large quantities of single stranded DNA. In this process a circularizable oligonucleotide is annealed and ligated to a template to form a circular DNA synthesis substrate. Upon addition of primer, deoxynucleotide triphosphates (dNTP's), and a strand displacing DNA polymerase, a single stranded product composed of multiple repeating copies of the circular substrate is produced. Coded within the sequence of the circular substrate are one or more binding sites (specific target sequence(s)) for the cleavage probe. As product is generated, increasing numbers of sites/specific target sequence(s) become available for binding of the probe and cleavage of the RNA moiety by RNase H, after which the probe dissociates and the cycle is repeated. After dissociation, the two fluorescently labeled DNA segments diffuse away from each other, increasing the distance between fluorescein and the TAMRA quencher, with the increase in fluorescein emission being monitored. The end result is a process in which the cyclic detection phase is coupled to DNA amplification of the circular substrate. Since the circularizable substrate is in excess over the template, assay sensitivity can be determined by varying the amount of template present in the reaction. FIG. 8 shows the results of such an assay in which either undiluted (▪), 1:10 (♦), 1:10² (▴), 1:10³ (●), 1:10⁴ (□), or 1:10⁵ (⋄) 10-fold serial dilutions of circularized template were amplified by RCA in the presence of the probe at 37° C. The control reaction (Δ) was performed with undiluted substrate in the absence of DNA polymerase. These results demonstrate that the cleavage probe can be used to monitor the real-time products of RCA amplification in a concentration dependent manner using the method of the present invention.

### EXAMPLE 4

Detection of Single Nucleotide Polymorphisms with the Fluorogenic Probe and RNase H.

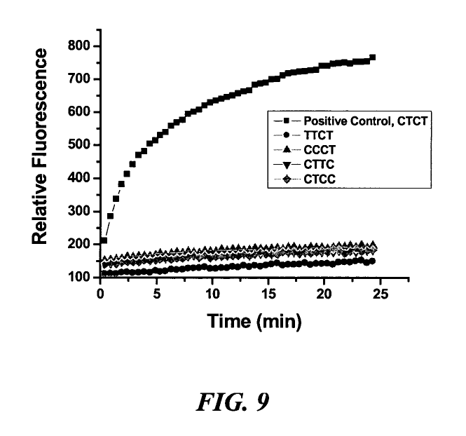

Referring now to FIG. 9, the ability of RNase H to cleave target sequences with a single base pair mismatch within the RNA hybridizing portion of the target sequence is shown. Four mismatch target DNA oligonucleotides were synthesized. These oligonucleotides are complementary to the probe except for the one mismatch. For example, oligonucleotide 1C to 1T indicates that only the corresponding complementary sequence for the first 5′ RNA nucleotide on the probe has been changed from a C to a T. 20 pmol of each of the mismatch target nucleotides were incubated with 10 pmol of fluorescent probe and 5 units of thermostable RNase H in 50 μl of RNase H buffer and monitored for 25 min. at 50° C. The results demonstrate that even a single nucleotide mismatch results in the absence of cleavage and corresponding increase in fluorescence intensity. These results further exemplify the extreme specificity that is provided by the reaction. Hence, the method by itself or in conjunction with a nucleic acid amplification reaction is an extremely powerful tool to detect single nucleotide polymorphisms.

It is understood that the specific order or hierarchy of steps in the method(s) disclosed are examples of exemplary approaches. Based upon design preferences, it is understood that the specific order or hierarchy of steps in the method(s) can be rearranged while remaining within the scope and spirit of the present invention. The accompanying method claims present elements of the various steps in a sample order, and are not necessarily meant to be limited to the specific order or hierarchy presented.

It is believed that the present invention and many of its attendant advantages will be understood by the forgoing description. It is also believed that it will be apparent that various changes may be made in the form, construction and arrangement of the components thereof without departing from the scope and spirit of the invention or without sacrificing all of its material advantages. The form herein before described being merely an explanatory embodiment thereof. It is the intention of the following claims to encompass and include such changes.

1. A method for real-time detection of a target nucleic acid, comprising:
(a) forming a reaction mixture of a target nucleic acid sequence and a plurality of nucleic acid probes which each include an enzyme mediated cleavable sequence and a detectable marker under conditions wherein a first nucleic acid probe of the plurality of nucleic acid probes including a first enzyme mediated cleavable sequence and a first detectable marker is allowed to hybridize to the target nucleic acid sequence creating a target-probe complex;
(b) contacting the target-probe complex with a cleaving agent which cleaves the first nucleic acid probe at a cleaving site within the enzyme mediated cleavable sequence forming a first nucleic acid probe fragment and a second nucleic acid probe fragment wherein the first and second nucleic acid probe fragments dissociate from the target nucleic acid;
(c) repeating steps (a) and (b) utilizing secondary nucleic acid probes from the plurality of nucleic acid probes within the reaction mixture, wherein a plurality of dissociated nucleic acid probe fragments are formed; and
(d) detecting the detectable markers activated by the dissociation of the plurality of nucleic acid probe fragments, thereby detecting the target nucleic acid.

2. The method of claim 1, wherein the enzyme mediated cleavable sequence is at least one of a ribonucleic acid (RNA) and a deoxyribonucleic acid (DNA).

3. The method of claim 1, wherein the cleaving site is located in a position which allows for the activation of the detectable marker upon cleavage of the probe.

4. The method of claim 1, wherein the plurality of nucleic acid probes further include a first probe region and a second probe region connected with the enzyme mediated cleavable sequence.

5. The method of claim 4, wherein the first probe region is at least one of a ribonucleic acid (RNA) and a deoxyribonucleic acid (DNA) and the second probe region is at least one of a ribonucleic acid (RNA) and a deoxyribonucleic acid (DNA).

6. The method of claim 4, wherein at least one of the enzyme mediated cleavable sequence, the first probe region, and the second probe region is at least one of fully methylated and partially methylated to prevent non-specific cleavage.

7. The method of claim 1, wherein the detectable marker is at least one of attached at the 5′ end of the first probe region, 3′ end of the first probe region, 5′ end of the second probe region, 3′ end of the second probe region, internally within either the first probe region or second probe region, 5′ end of the enzyme mediated cleavable sequence, 3′ end of the enzyme mediated cleavable sequence, and internally within the enzyme mediated cleavable sequence.

8. The method of claim 1, wherein the detectable marker is selected from the group consisting of a fluorescent molecule, radioisotopes, enzymes, or chemiluminescent catalysts.

9. The method of claim 1, wherein the detectable marker is at least one of an internally labeled Forster resonance energy transfer (FRET) pair, externally labeled FRET pair, and a FRET pair attached at a 3′ end of the first probe region and a 5′ end of the second probe region.

10. The method of claim 1, wherein the cleaving agent is selected from the group consisting of an an RNase H, an Kamchatka crab duplex specific nuclease, an endonuclease, an nicking endonuclease, an exonuclease, or an enzyme containing nuclease activity.

11. The method of claim 1, wherein the target nucleic acid is at least one of a ribonucleic acid (RNA) and a deoxyribonucleic acid (DNA).

12. The method of claim 1, wherein the steps of the method occur during a process for amplifying the target nucleic acid.

13. The method of claim 12, wherein the process for amplifying the target nucleic acid is selected from the group consisting of rolling circle amplification, polymerase chain reaction, nucleic acid sequence based amplification, or strand displacement amplification.

14. The method of claim 1, wherein the detection of probe fragments is performed in at least one of real-time and post-reaction.

15. A method for real-time detection of a target epitope, comprising:
(a) obtaining a target eptiope;
(b) preparing an aptamer having an attached target nucleic acid sequence being complementary to a first nucleic acid probe including a first enzyme mediated cleavable sequence and a first detectable marker;
(c) hybridizing the aptamer to the target epitope, forming a complex;
(d) forming a reaction mixture of a plurality of nucleic acid probes each having an enzyme mediated cleavable sequence and detectable marker and the target nucleic acid sequence under conditions allowing the hybridization of the first nucleic acid probe of the plurality of nucleic acid probes including the first enzyme mediated cleavable sequence and first detectable marker to the target nucleic acid sequence creating a target nucleic acid-probe complex;
(e) contacting the target nucleic acid-probe complex with a cleaving agent which cleaves the first probe at a cleaving site within the enzyme mediated cleavable sequence forming a first probe fragment and a second probe fragment wherein the first and second probe fragments dissociate from the target nucleic acid;
(f) repeating steps (d) and (e) utilizing secondary nucleic acid probes from the plurality of nucleic acid probes within the reaction mixture, wherein a plurality of dissociated probe fragments are formed; and
(g) detecting the detectable markers activated by the dissociation of the plurality of probe fragments, thereby detecting the target epitope.

16. The method of claim 15, wherein the aptamer includes at least one of a single aptamer, two or more aptamers, and three or more aptamers.

17. The method of claim 15, wherein the epitope is bound with specificity by an antibody attached with the target nucleic acid sequence, wherein the antibody is at least one of a monoclonal antibody and a polyclonal antibody.

18. The method of claim 17, wherein more than one target nucleic acid sequence is attached to at least one of the monoclonal antibody and polyclonal antibody.

19. The method of claim 15, wherein the enzyme mediated cleavable sequence is at least one of a ribonucleic acid (RNA) and a deoxyribonucleic acid (DNA).

20. The method of claim 15, wherein the cleaving site is located in a position which allows for the activation of the detectable marker upon cleavage of the probe.

21. The method of claim 15, wherein the plurality of nucleic acid probes further include a first probe region and a second probe region connected with the enzyme mediated cleavable sequence.

22. The method of claim 21, wherein the first probe region is at least one of a ribonucleic acid (RNA) and a deoxyribonucleic acid (DNA).

23. The method of claim 21, wherein the second probe region is at least one of a ribonucleic acid (RNA) and a deoxyribonucleic acid (DNA).

24. The method of claim 21, wherein at least one of the enzyme mediated cleavable sequence, the first probe region, and the second probe region is at least one of fully methylated and partially methylated to prevent non-specific cleavage.

25. The method of claim 15, wherein the cleaving agent is selected from the group consisting of an RNase H, an Kamchatka crab duplex specific nuclease, an endonuclease, an nicking endonuclease, an exonuclease, or an enzyme containing nuclease activity.

26. The method of claim 15, wherein the detectable marker is at least one of attached at the 5′ end of the first probe region, 3′ end of the first probe region, 5′ end of the second probe region, 3′ end of the second probe region, internally within either the first probe region or second probe region, 5′ end of the enzyme mediated cleavable sequence, 3′ end of the enzyme mediated cleavable sequence, and internally within the enzyme mediated cleavable sequence.

27. The method of claim 15, wherein the detectable marker is selected from the group consisting of fluorescent molecules, fluorescent antibodies, radioisotopes, enzymes, proteins, or chemiluminescent catalysts.

28. The method of claim 27, wherein the detectable marker is at least one of an internally labeled Förster resonance energy transfer (FRET) pair, externally labeled FRET pair, and a FRET pair attached at a 3′ end of the first probe region and a 5′ end of the second probe region.

29. The method of claim 15, wherein the target nucleic acid is at least one of a ribonucleic acid (RNA) and a deoxyribonucleic acid (DNA).

30. The method of claim 15, wherein the steps of the method occur during a process for amplifying the target nucleic acid.

31. The method of claim 30, wherein the process for amplifying the attached target nucleic acid sequence is selected from the group consisting of rolling circle amplification, polymerase chain reaction, nucleic acid sequence based amplification, or strand displacement amplification.

32. The method of claim 15, wherein the detection of probe fragments is performed in at least one of real-time and post-reaction.

33. A method for real-time detection of a single nucleotide polymorphism within a target nucleic acid, comprising:
(a) forming a reaction mixture of a target nucleic acid sequence including a single nucleotide polymorphism and a plurality of nucleic acid probes which each include an enzyme mediated cleavable sequence and detectable marker under conditions wherein a first probe of the plurality of nucleic acid probes including a first enzyme mediated cleavable sequence and a first detectable marker is allowed to hybridize to the target nucleic acid sequence creating a target-probe complex;
(b) contacting the target-probe complex with a cleaving agent which cleaves the first nucleic acid probe at a cleaving site within the enzyme mediated cleavable sequence forming a first nucleic acid probe fragment and a second nucleic acid probe fragment wherein the first and second nucleic acid probe fragments dissociate from the target nucleic acid;
(e) repeating steps (a) and (b) utilizing secondary probes from the plurality of nucleic acid probes within the reaction mixture, wherein a plurality of dissociated nucleic acid probe fragments are formed; and
(c) detecting the detectable markers activated by the dissociation of the plurality of nucleic acid probe fragments, thereby detecting the single nucleotide polymorphism of the target nucleic acid sequence.

34. The method of claim 33, wherein the detectable marker is selected from the group consisting of fluorescent molecules, fluorescent antibodies, radioisotopes, enzymes, proteins, or chemiluminescent catalysts.

35. The method of claim 33, wherein the cleaving site is located in a position which allows for the activation of the detectable marker upon cleavage of the probe.

36. The method of claim 33, wherein the steps of the method occur during a process for amplifying the target nucleic acid sequence.

37. The method of claim 36, wherein the process for amplifying the target nucleic acid sequence is selected from the group consisting of rolling circle amplification, polymerase chain reaction, nucleic acid sequence based amplification, or strand displacement amplification.

38. The method of claim 33, wherein the cleaving agent is selected from the group consisting of an RNase H, DNases, RNases, helicases, exonucleases, restriction endonucleases, and endonucleases.

39. The method of claim 33, wherein the detection of probe fragments is performed in at least one of real-time and post-reaction.

40. The method of claim 33, wherein the hybridization of a nucleic acid probe to a target nucleic acid sequence, the target nucleic acid including a single nucleotide polymorphism, contains a base pair mismatch, resulting in the probe remaining hybridized to the target nucleic acid sequence after contact with the cleaving agent.

41. The method of claim 12, wherein the steps of the method occur under non-isothermic conditions.

42. The method of claim 30, wherein the steps of the method occur under non-isothermic conditions.

43. The method of claim 36, wherein the steps of the method occur under non-isothermic conditions.


SEQUENCE LISTING (4 sequences)

SEQ ID NO: 1 - DNA, 24 bp, Artificial Sequence
  Synthesized construct wherein positions 11 through 14 comprise the RNA section of the sequence.
tatgccatttgagatttttgaatt

SEQ ID NO: 2 - DNA, 60 bp, Artificial Sequence
  Synthesized construct of a 60-mer oligonucleotide template for facilitating simultaneous cleavage probe/rolling circle amplification assay to detect DNA.
atctgactatgcttgtacctggttatttagcactcgtttttaatcagctcactagcacct

SEQ ID NO: 3 - DNA, 80 bp, Artificial Sequence
  Synthesized construct of a 80-mer circularizable oligonucleotide template for facilitating simultaneous cleavage probe/rolling circle amplification assay to detect DNA.
ctaaataaccaggtacaatatgccatttgagatttttgaattggtcttagaacgccattttggctgattaaaaacgagtg

SEQ ID NO: 4 - DNA, 15 bp, Artificial Sequence
  Synthesized construct of a 15-mer oligonucleotide primer for facilitating simultaneous cleavage probe/rolling circle amplification assay to detect DNA.
tggcgttctaagacc